(19) United States
(12) Patent Application Publication
(10) Pub. No.: US 2009/0302498 A1
(43) Pub. Date: Dec. 10, 2009

(54) SENSING DEVICE

(75) Inventor: Stefan Nedestam, Molnlycke (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 12/514,096

(22) PCT Filed: Dec. 18, 2006
(86) PCT No.: PCT/SE2006/001436

§ 371 Date: May 8, 2009

(51) Int. Cl. B29C 39/12; A61F 13/42

(52) U.S. Cl. 264/263; 604/361

(57) ABSTRACT

A sensing device including a magnetoelastic film and a polymeric support for the magnetoelastic film, the magnetoelastic film being mounted in the support. The polymeric support includes a mixture of polymeric material and magnetically-susceptible material, the magnetically-susceptible material being in particulate form. A method for the production of the sensing device, and an absorbent article including the sensing device.

### TECHNICAL FIELD

The present invention relates to a sensing device (sensor) which is primarily used for detecting wetness or moisture in a given environment. The invention also relates to a method for the production of said sensing device, the use of said sensing device in an absorbent article and an absorbent article comprising said sensing device.

### BACKGROUND OF THE INVENTION

It is known to provide disposable absorbent articles, such as diapers and incontinence articles, with sensors of some sort, which allow the wearer or caregiver to readily determine the status of the article, particularly how wet or dry it is. In this way, the carer or wearer is alerted to a soiling incident, and unnecessary changes of the article can be avoided.

It is known to utilize elements comprising a magnetoelastic material for different types of detections or measurements within a variety of technical fields. For example, elements of a magnetoelastic material are used in position sensors, identification markers and in anti-theft tags or electronic article surveillance (EAS) tags. In addition, it is known to use elements of a magnetoelastic material in sensors for detection or measurement of environmental parameters. Grimes et al. (Biomedical Microdevices, 2: 51-60, 1999) have described sensors comprising an element of a magnetoelastic material, i.e. magnetoelastic sensors, and their use in measurements of e.g. temperature, pressure or viscosity.

Furthermore, it is known to include magnetoelastic sensors in absorbent articles, such as diapers, pant-type diapers, incontinence garments, sanitary napkins, bed protectors, wipes, towels, tissues, tampon-like products and wound or sore dressings, which absorbent articles are intended to be utilized for absorption, retention and isolation of body wastes, such as urine, faeces and blood. A magnetoelastic sensor comprised in such an absorbent article may be designed to respond to an event, such as urination or defecation, after absorption onto or into the absorbent article. The response may be, for example, a signal after the event has occurred and may be based on measurement of, for example, wetness, a biological analyte and/or a chemical analyte. The signal of an event enables the user, parent, caregiver, nursing personnel, etc. to determine with ease that an event has occurred.

WO 2004/021944 describes a disposable sensoring absorbent structure comprising at least one absorbent layer and at least one sensing device comprising a magnetoelastic film. The sensoring absorbent structure may be comprised in an absorbent article such as, for example, a diaper, pant-type diaper, an incontinence protector, a sanitary napkin or a bed protector. In one embodiment, the sensing device is intended to be utilized for detection of wetness. The magnetoelastic film of the sensing device is then coated with a wetness sensitive polymer which interacts with wetness, e.g. moisture, a liquid or humidity. The wetness sensitive polymer interacts with wetness, such as urine, through absorption or adsorption, whereby the mass of the sensing device changes. This change in mass will either increase or decrease the resonant frequency of the magnetoelastic film. The mass change is measurable and correlates to the amount of wetness interacting with the wetness sensitive polymer. In another embodiment, the magnetoelastic film of the sensing device is coated directly or indirectly with at least one detector molecule adapted to detect at least one target biological and/or chemical analyte in body waste, body exudates or the user's/wearer's skin. WO 2004/021944 is herein incorporated by reference in its entirety.

It is possible to excite the magnetoelastic material with a continuous magnetic field showing a frequency corresponding to the magnetoacoustic resonant frequency and measure the response from the material. At this resonant frequency the response from the material is maximal. Hence, a permanent bias magnet is mounted in such sensing devices, and is arranged such that its magnetic field lies parallel to the plane of the magnetoelastic film.

In order to obtain the relatively small dimensions which are required for these sensing devices (ca. 3-20 mm wide, ca. 10-40 mm long and ca. 1-5 mm thick), these bias magnets have traditionally been manufactured from a ribbon or tape of magnetic material, from which magnets of the correct dimensions have been cut or otherwise formed.

This approach suffers from a number of disadvantages, however. The magnetic field in the magnets obtained by this method is rather irregular, due to differences in the cutting process and small defects in the edges of each magnet. Difficulties also arise in positioning the magnetoelastic material with respect to the bias magnet—as the magnetic field of the bias magnet is not uniform, a small displacement of the magnetoelastic material with respect to the magnet can have significant effects on the field experienced by the magnetoelastic material. This problem is discussed in WO/00/02172.

Furthermore, cutting out a magnet, positioning it and mounting it in a sensing device involves three process steps, which are additionally complicated by the relatively small size of the components involved.

In addition, absorbent articles used in the hygiene field must meet strict requirements in terms of product safety, as they are often in contact or near proximity with the body of the wearer in conditions which are usually warm and may also be damp. It is therefore important that the components of any sensing device are able to withstand such environments without promoting allergies in the wearer, for example. The ribbons or tapes of magnetic material, while being suitable for applications such as fridge magnets, are not always suitable for hygiene applications, as their composition is often indeterminate, they may contain allergenic or toxic components and they may leach chemicals into the surrounding absorbent article.

There is therefore a need for an improved sensing device which avoids the problems associated with the above-described sensing devices. In particular, a sensing device is required which comprises a bias magnet having a reproducible, more homogeneous magnetic field and which can be manufactured with ease. Moreover, it is desirable to obtain a sensing device in which the magnet meets the strict requirements of product safety for use in hygiene products.

### SUMMARY OF THE INVENTION

In a first embodiment, therefore, the present invention provides a sensing device, comprising a magnetoelastic film and a polymeric support for said magnetoelastic film, said magnetoelastic film being mounted in said support. The polymeric support comprises a mixture of polymeric material and magnetically-susceptible material. The magnetically-susceptible material is in particulate form.

The present invention also provides a method for producing a sensing device as described herein, said method comprising the steps of:
a. mixing polymeric material with magnetically-susceptible material in particulate form;
b. casting a polymeric support from the mixture of polymeric material and magnetically-susceptible material;
c. magnetising the polymeric support so as to provide it with an overall magnetic field B;
d. mounting the magnetoelastic film on the polymeric support such that the plane of the magnetoelastic film is oriented in a direction which is parallel to the magnetic field B.

The present invention also describes the use of one or more sensing devices as described herein for detecting wetness or moisture in an absorbent article, such as a diaper, incontinence guard, sanitary napkin or panty liner. The present invention also provides an absorbent article, such as a diaper, incontinence guard, sanitary napkin or panty liner, comprising one or more sensing devices as described herein.

### BRIEF DESCRIPTION OF THE DRAWINGS

FIG. 1 shows an exploded view of a sensing device according to the prior art.

FIG. 2 shows an exploded view of a sensing device according to the present invention. The insert in FIG. 2 is a magnified cross-section of the sensing device of FIG. 2 as shown.

FIG. 3 shows an absorbent article comprising a sensing device according to the invention.

FIG. 4 shows resonance frequency and amplitude versus the magnetic bias field.

FIG. 5 shows the magnetic field from the bias magnet at the location of the MER film.

### DETAILED DESCRIPTION OF PREFERRED EMBODIMENTS

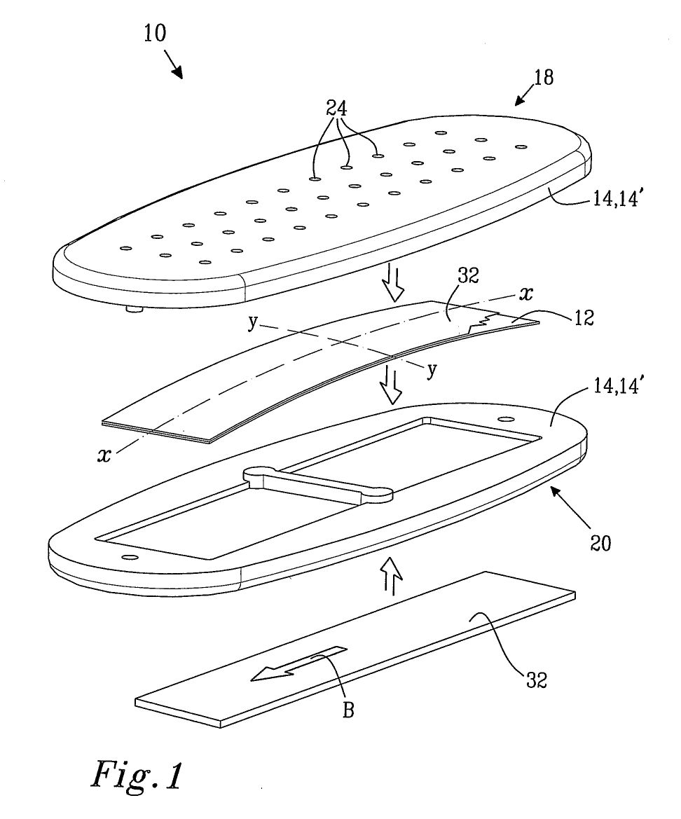

FIG. 1 shows a sensing device 10 according to the prior art. It comprises a strip-shaped magnetoelastic film 12 mounted in a polymeric support 14. The polymeric support 14 is in the form of a capsule 14′ which has a hollow interior and which completely encloses the magnetoelastic film 12. The capsule 14′ has a lid 18 and a base 20. The center of the magnetoelastic film 12 is mounted in the middle of the base 20 in the longitudinal direction (x) and fixed in place with adhesive, allowing the ends of the magnetoelastic film 12 to move freely. A permanent bias magnet 32 is mounted in the underside of the base 20 of the sensing device 10, and is arranged such that its magnetic field B lies parallel to the plane of the magnetoelastic film.

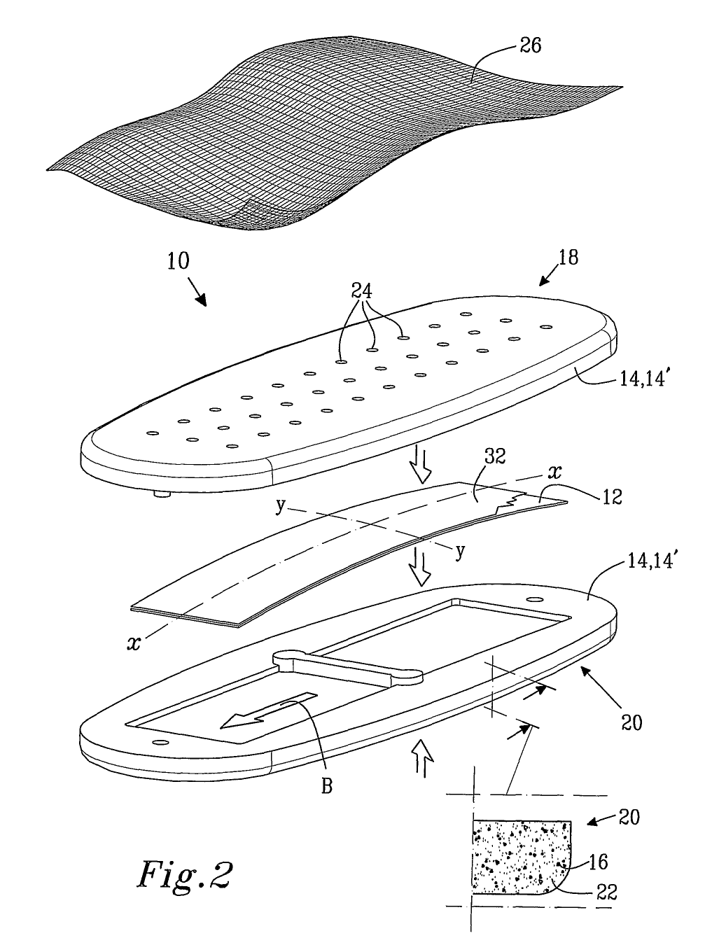

The present invention provides a sensing device 10, illustrated generally in FIG. 2. It comprises a strip-shaped magnetoelastic film 12 mounted on a polymeric support 14. As described above, the sensing device 10 can be placed in an absorbent article and be used to detect the presence of moisture.

### Magnetoelastic Materials

The magnetoelastic film 12 of the sensing device 10 is typically strip-shaped, with a longitudinal axis (x) and a transverse axis (y), and with a greater extension in the longitudinal direction than the transverse direction. Other shapes for the magnetoelastic film, such as e.g. square, are also possible.

Materials that are suitable for use as the magnetoelastic film 12 in a magnetoelastic sensor are materials with a non-zero magnetostriction and a high magnetoelastic coupling such as, for example, iron-nickel alloys, rare earth metals, ferrites, e.g. spinel type ferrites (Fe₃O₄, MnFe₂O₄), silicon-iron alloys, many other different alloys and mixtures thereof. Soft magnetoelastic materials, alloys and mixtures thereof as well as amorphous magnetoelastic materials, alloys and mixtures thereof may be used. Examples of amorphous magnetoelastic alloys are metglases such as Fe₄₀Ni₃₈Mo₄B₁₈, e.g. Metglas 2826MB3 (Honeywell Amorphous Metals, Pittsburg, Pa., USA), Metglas 2826MB2 (FeCo)₈₀B₂₀, (CoNi)₈₀B₂₀ and (FeNi)₈₀B₂₀. The film of magnetoelastic material is typically about 0.01-1000 μm, such as 0.01-200 μm, 5-100 μm or 0.01-100 μm thick.

The term “magnetostriction” refers to a phenomenon whereby a material changes its dimensions in the presence of an external magnetic field. The extent of the dimensional change depends on the magnetization in the material and, of course, on the material properties. The phenomenon of magnetostriction is due to the interaction between the atomic magnetic moments in the material.

A material having a “high magnetoelastic coupling” efficiently converts magnetic energy into mechanical elastic energy and vice versa. When a material that can convert magnetic energy into mechanical elastic energy is excited by a varying magnetic field, elastic waves mechanically deform the material, which has a mechanical resonant frequency inversely proportional to its length. If the material also is magnetostrictive, it generates a magnetic flux when the material is mechanically deformed. This magnetic flux extends remotely and may be detected by a pick-up coil.

Furthermore, a magnetoelastic material stores magnetic energy in a magnetoelastic mode when excited by an external magnetic field. When the magnetic field is switched off, the magnetoelastic material shows damped oscillation with a specific resonant frequency denoted as the magnetoacoustic resonant frequency. These oscillations give rise to a magnetic flux that varies in time, which can be remotely detected by a pick-up coil. If a pulsed magnetic field such as, for example, a pulsed sine-wave magnetic field, is applied to the magnetoelastic material, it will be possible to detect this characteristic resonant frequency between the magnetic pulses. The resonant frequency is inversely proportional to the length of the piece of magnetoelastic material.

The pulse frequencies used may be, for example, about 10-1000 Hz, such as about 50-700 Hz. The duty cycles of the pulses may be, for example, about 1-90%, such as about 10-50%. If the magnetic field is a pulsed sine wave field, the sine waves may be, for example, about 50-80 kHz. If METGLAS® material from Honeywell is used as the magnetoelastic material, a magnetic field amplitude of the pulsing field may be about 0.05-0.1 mT.

An excitation coil may, for example, be used for applying a magnetic field to the magnetoelastic film. The pick-up coil may be used to collect the produced signal, i.e. for detecting the resonant frequency. The excitation coil and the pick-up coil may be located in a handheld unit, preferably in the same handheld unit. In a further alternative, the same coil may be utilized as both excitation coil and pick-up coil, i.e. it may be utilized for both excitation and detection. WO 2004/021944 is herein incorporated by reference in its entirety for further details regarding the excitation of the magnetoelastic material, detection of the resonant frequency and devices for the excitation and detection.

The magnetoelastic film 12 is coated with a layer comprising a sensing material 32 arranged to interact with an analyte. For example, the magnetoelastic film 12 may be coated with a sensing polymer such as e.g. a wetness sensitive polymer. In one embodiment, the sensing material 32 is a wetness sensitive polymer selected from the group consisting of linear and hydrophilic polymers or chemically/physically cross-linked swellable polymer gels based on polyvinyl alcohol, polyvinyl pyrrolidone, polyethylene oxide and co-polymers thereof, polyurethane, polyamides, starch and derivatives thereof, cellulose and derivative thereof, polysaccharides, proteins, polyacrylonitrile, polyethylene imine, acrylate based polymers, and mixtures thereof. Polyalcohol-based polymer gels, such as polyvinyl alcohol-based polymer gels are preferred sensing materials 32 for the detection of moisture. The thickness of the layer of sensing material 32 may be, for example, about 0.005-500 μm, such as 0.005-100 μm, 2.5-50 μm, 5-50 μm or 34 μm. An example of a magnetoelastic sensor in which the magnetoelastic material is coated with a sensing material is disclosed in WO 2004/021944.

Alternatively, the magnetoelastic film 12 may be coated with detector molecules or may be coated with a non-wetness sensitive polymer, which in turn is coated with detector molecules. The detector molecules are adapted to detect at least one target biological and/or chemical analyte.

The detector molecule may in one variant be adapted to detect a biological or chemical analyte selected from the group consisting of an enzyme or a sequence of enzymes; an antibody; a nucleic acid, such as DNA or RNA; a protein, such as a soluble protein or a membrane protein; a peptide, such as an oligopeptide or a polypeptide; an organelle; parts of a natural or synthetic cell membrane or capside, such as a bacterial or a mammalian cell membrane, or a virus capside; an intact or partial viable or nonviable bacterial, plant or animal cell; a piece of plant or mammalian tissues or any other biologically derived molecule; a lipid, a carbohydrate; a lectin, and mixtures thereof.

In another variant, the detector molecule may be adapted to detect a biological or chemical analyte selected from the group consisting of pathogenic bacteria; non-pathogenic bacteria, e.g. colonic bacteria; viruses; parasites; bacterial toxins; fungi; enzymes; proteins; peptides; mammalian blood cells, such as human white or red blood cells; hormones; mammalian, including human, blood components, such as blood glucose; urine and its components such as glucose, ketones, urobilinogen, and bilirubin; and mixtures thereof.

The bacteria that the detector molecule may be adapted to detect, pathogenic or not, is selected from the group consisting of Escherichia coli, Salmonela typhi, Salmonella paratyphi, Salmonella enteriditid, Salmonella thyphimurium, Salmonella heidelberg, Staphylococcus aureus, Shigella sonnei, Shigella flexneri, Shigella boydii, Shigella dysenteriae, Vibrio cholerae, Mycobacterium tuberculosis, Yersina enterocolitica, Aeromonas hydrophila, Plesmonas shigelloides, Campylobacter jejuni, Campylobacter coli, Bacteroides fragilis, Clostridia septicum, Clostridia perfringens, Clostridia botulinum, Clostridia difficile, and mixtures thereof.

In still another variant, the detector molecule is adapted to detect a chemical compound or chemical analyte such as health markers or nutritional markers. Nutritional markers include markers for e.g. metabolic efficiency, nutrient deficiencies, nutrient absorption or malabsorption, food and drink intake, food allergies (e.g. to peanuts), food intolerance (e.g. lactose or gluten intolerance), colonic bacteria ecology (e.g. beneficial bacterias such as bifidobacteria and lactobacillus), and total energy balance. Health markers may include chemical analytes such as heavy metals (e.g. lead, mercury, etc.), radioactive substances (e.g. caesium, strontium, uranium, etc.), fats, enzymes, endogenous secretions, protein matter (e.g. blood casts), mucous, and micro-organisms, as described above, that may be related to various health issues such as infection, diarrhoea, gastrointestinal distress of disease, or poisoning. Heavy metals, especially in certain developing countries and in older and/or less affluent areas of developed countries, are serious health risks. For example, lead and mercury poisoning may occur upon the ingestion of these heavy metals from environmental sources (e.g. from lead paint, unregulated heavy industries, etc.) and can be fatal. More commonly, low-level poisoning by these and other heavy metals results in retarded intellectual and/or physical development, especially in children that may occur over a long time and have lasting effects on the individual. Other examples of nutritional markers include calcium, vitamins (e.g. thiamine, riboflavin, niacin, biotin, folic acid, pantothenic acid, absorbic acid, vitamin E, etc.), electrolytes (e.g. sodium, potassium, chlorine, bicarbonate, etc.), fats, fatty acids (long and short chain), soaps (e.g. calcium palmitate), amino acids, enzymes (e.g. lactose, amylase, lipase, trypsin, etc.), bile acids and salts thereof, steroids, and carbohydrates. For example, calcium malabsorption is important in that it may lead to a long-term bone-mass deficiency.

Suitable detector molecules may include any biorecognition element and are further exemplified by carbohydrates, antibodies or parts thereof, synthetic antibodies or parts thereof, enzymes, lectins, DNA (deoxyribonucleic acid), RNA (ribonucleic acid), cells and/or cell membranes or any other molecule with a binding capacity for a defined bioanalyte or chemical analyte.

Any suitable means or methods for applying the detector molecules to the mangetoelastic layer may be utilized. For example, it may be desirable to chemically bind the detector molecule, directly or indirectly, to the magntoelastic layer using any one of a variety of common crosslinker molecules including, but not limited to, glutaraldehyde, N-hydroxysuccinimide, carbodidimides.

Magnetoelastic films 12 that may be used in magnetoelastic sensing devices are typically produced as a continuous ribbon. In production of magnetoelastic films 12 in which the magnetoelastic material is coated with a layer comprising a sensing material 32, a ribbon of a magnetoelastic film 12 is typically coated with the sensing material 32 and the coated ribbon is thereafter divided into strips forming different sensors. However, the ribbon may likewise firstly be divided into strips and the sensing material 32 may thereafter be coated on the separated strips.

As described above, the magnetoelastic film 12 oscillates at a specific resonant frequency in its initial state—i.e. before exposure to water vapour or chemical analyte. When the magnetoelastic film 12 and coating of sensing material 32 is exposed to water vapour or chemical analyte, they bind to the surface of the magnetoelastic film 12 via the sensing material 32. As a result, the mass of the magnetoelastic film 12 increases, which leads to a measurable change in the resonant frequency, which in turn can be detected by the pick-up coil.

### Polymeric Support

The sensing device 10 of the invention comprises a polymeric support 14 for the magnetoelastic film 12. The magnetoelastic film 12 is fixedly mounted in at least one position on said support 14, so that the magnetoelastic film 12 can vibrate. Like any vibrating structure, the magnetoelastic film 12 has vibrational nodes at which displacement is zero. Most commonly, there is a vibrational node at the centre of the magnetoelastic film 12, so the most suitable position for the magnetoelastic film 12 to be supported is only in the centre of the magnetoelastic film 12 as measured in the longitudinal direction (x).

The polymeric support 14 comprises a mixture of polymeric material 22 and magnetically-susceptible material 16 (see FIG. 2, inset). The polymeric material 22 can be any known polymeric material 22 which meets the requirements of strength, flexibility, durability, stability and mouldability which are needed in a sensing device 10. The polymeric material 22 is usually in particulate or granular form before being mixed with the magnetically-susceptible material 16 and processed. The polymeric material 22 may be copolymer or block copolymer, and may be synthetic or natural. Polyamines, polyamides, polyethers, polyalkenes (e.g. polyethylene), polyacrylates, polyacrylamides, polystyrenes, polyvinylhalides and polyesters, and mixtures thereof are all suitable polymeric materials for use in the present invention. Polyamides and acrylonitrile-butadiene-styrene (ABS) copolymer function well from a manufacturing point of view, and are approved for use in hygiene applications.

Typically, the polymeric support 14 is formed through a moulding technique such as injection moulding, compression moulding, transfer moulding, extrusion moulding, blow moulding, rotational moulding (rotomoulding), thermoforming, vacuum forming (a simplified version of thermoforming), reaction injection moulding, laminating, expandable bead moulding or foam moulding. The polymeric support 14 can also be formed through extrusion processes.

The polymeric support 14 illustrated in FIG. 2 is in the form of a capsule 14′ which has a hollow interior and which completely encloses the magnetoelastic film 12. The capsule 14′ has a lid 18 and a base 20. The magnetoelastic film 12 is mounted in the middle of the base 20 and fixed in place (e.g. with adhesive), allowing each end of the film 12 to move freely.

The capsule 14′ of FIG. 2 has an oval cross-section, with the boundary between lid 18 and base 20 running along the perimeter of the capsule 14′. However, the capsule 14′ may take other geometric forms, such as cylindrical, with the boundary between lid 18 and base 20 at one end or aligned with the major or minor axis of symmetry. Capsules 14′ with a cross-section which is rectangular, square or any other geometry are also possible. All these forms can be readily provided by the moulding techniques discussed above. Provided that the capsule 14′ offers adequate protection to the magnetoelastic film 12 and allows it to vibrate freely, and at the same time allows generation of a homogeneous magnetic field B, the precise geometry of the capsule is not of major significance.

The capsule 14′ comprises at least one opening 24 which allows the passage of gases into the capsule 14′. In FIG. 2, these openings 24 are illustrated as a plurality of small through-holes in the lid 18, although alternative arrangements of the openings 24 are possible, e.g. slots or open areas in the lid 18 or base 20, or discontinuities in the boundary between lid 18 and base 20. The capsule 14′ may also have a relatively “open” structure in which the openings 24 are comparatively large.

In a preferred embodiment, the at least one opening 24 in the capsule 14′ is covered with a liquid-impermeable, gas-permeable layer 26. This limits the access of liquid to the interior of the capsule 14′—which might otherwise destroy or reduce the effect of the magnetoelastic film 12—but allows vapour to pass in and out of the capsule. If desired, the entire capsule 14′ may be covered with the liquid-impermeable, gas-permeable layer 26. Suitable liquid-impermeable, gas-permeable layers 26 are nonwoven materials, perforated plastic films and laminates thereof.

### Magnetically-susceptible Material

The polymeric support 14 of the invention comprises a mixture of the polymeric material 22 and magnetically-susceptible material 16. This is illustrated schematically in the magnified inset in FIG. 2. The term “magnetically-susceptible material” refers to a material which becomes magnetised when subjected to a magnetic field. The magnetic susceptibility of a material is the degree of magnetization of said material in response to an applied magnetic field.

The magnetically-susceptible material 16 is in particulate form. In the present context, the expression “particulate form” includes forms such as granules and powders, and describes a material in which the average diameter of the particles lies between 1 and 4 μm. The magnetically-susceptible material 16 is mixed with the polymeric material 22 to form a substantially homogeneous mixture.

Injection-moulded magnets comprise a magnetic powder which is dispersed in a polymer, and allow complex shapes to be manufactured by injection moulding. To promote binding and mixing between the magnetic powder and the polymer, the magnetic powder is often chemically altered in some way, and may comprise polymerisable groups which are incorporated into the polymer upon polymerisation. The physical and magnetic properties of injection-moulded magnets depend on the raw materials, but they generally have a lower magnetic strength and resemble plastics in their physical properties. U.S. Pat. No. 4,358,388 describes magnetic polymer latex and a process for its preparation.

The magnetically-susceptible material 16 is preferably ferromagnetic with a residual magnetisation (remenance). Application of an external magnetic field causes a magnetisation of the magnetically-susceptible material, creating an overall magnetic field B. When the external magnetic field is switched off, the magnetic susceptible material should have a residual magnetisation that creates the magnetic field B inside the capsule. Suitable magnetically-susceptible materials are selected from the group comprising a metal, a metal alloy, iron oxide, chromium dioxide and mixtures of iron oxide and oxides of other metals. Suitable metals may be iron, iron-silicon, nickel or cobalt. Suitable metal alloys may be alloy of iron, iron-silicon, nickel or cobalt with at least one of molybdenum, chromium, copper, vanadium, manganese, strontium, aluminium and titanium. Iron oxides include Fe₃O₄ and γ-Fe₂O₃ or mixtures thereof. Iron oxides such as Fe₃O₄ and γ-Fe₂O₃ may be mixed with combined or mixed with other metal oxides such as cobalt oxide, manganese oxide, zinc oxide, barium oxide and rare earth oxides. Strontium ferrite and chromium oxide are magnetically-susceptible materials which may be used.

Ready-mixed blends of magnetically-susceptible material 16 in particulate form and polymeric material 22 are commercially available. For example, COMPODIC FPA-132 is a mixture of Strontium ferrite and polyamide 12, and is supplied by DIC Europe GmbH. YUXIANG Magnetic Materials Ind. Co., Ltd and Xiangying Magnetic Materials Co., Ltd are also suppliers of such ready-mixed products.

Suitably, the polymeric support 14 comprises between between 50 and 95 weight %, preferably between 75 and 90 weight % of the magnetically-susceptible material 16. Introduction of magnetically-susceptible material 16 into the polymeric support 14 can weaken the support 14 and make it brittle, so it is optimal for the strength and flexibility of the polymeric support 14 that only the lid 18 or the base 20 of the capsule 14′ comprises the magnetically-susceptible material 16. This also provides cost savings.

The magnetically-susceptible material 16 is usually in the form of particles, which are dispersed among the particles of the polymeric material 22 (see inset, FIG. 2). Preferably, the particles have an average diameter of between 0.5 and 3 mm, more preferably between 1 and 2.5 mm.

A magnetic field B is induced in the polymeric support 14, so that the field B is oriented in a direction which is parallel to the plane of the magnetoelastic film 12. In order to have the highest magnetic residual magnetisation (remanence) that gives the highest magnetic field inside the capsule, an external magnetic field of a predetermined level is applied in the longitudinal direction of the capsule. The minimum strength of this external magnetic field varies with dimension, geometry and magnetic material in the capsule, and is defined as where the two magnetisation branches in the magnetic hysteresis loop close.

If the magnetoelastic film 12 is strip-shaped, the magnetic field B of the polymeric support 14 is oriented in a direction which is parallel to the longitudinal axis (x) of the magnetoelastic film 12. This maximises the vibration in the magnetoelastic film 12 and thus maximises the strength of the signal emitted by the sensing device 10.

The strength of the magnetic field B in the polymeric support 14 lies between 0.1 and 2 mT, preferably between 0.1 and 0.5 mT.

By introducing magnetically-susceptible material 16 into the polymeric support 14 in this way, the need for a separate bias magnet 32, and the problems associated with such magnets, has been eliminated. Hence the sensing device 10 according to the invention does not contain a separate bias magnet 32; instead, the polymeric support 14 of the sensing device 10 itself acts as a bias magnet.

The present invention also concerns a method for producing a sensing device 10 as described herein, said method comprising the steps of:
a. mixing polymeric material 22 with magnetically-susceptible material 16;
b. casting a polymeric support 14 from the mixture of polymeric material 22 and magnetically-susceptible material 16;
c. magnetising the polymeric support 14 so as to provide it with an overall magnetic field B;
d. mounting the magnetoelastic film 12 on the polymeric support 14 such that the plane of the magnetoelastic film 12 is oriented in a direction which is parallel to the magnetic field B.

To eliminate variations in the resonant frequency of the sensing device 10 which might occur during manufacture, the method can further include the step of calibrating the sensing device 10 after the magnetoelastic film 12 has been mounted on the polymeric support 14.

As described above, the polymeric support 14 may comprises a capsule 14′, having a lid 18 and a base 20 and at least one opening 24 which allows the passage of gases. In this case, step d. (above) comprises the individual steps of mounting the magnetoelastic film 12 in the base 20 of the capsule 14′ such that the plane of the magnetoelastic film 12 is oriented in a direction which is parallel to the magnetic field B of the capsule 14′ and closing the lid 18 so that the magnetoelastic film 12 is completely enclosed within the capsule 14′.

### Absorbent Article

A sensing device 10 according to the invention may be positioned in contact with or in spaced relation with an absorbent material of an absorbent structure 34 of an absorbent article 30. For example, a sensing device 10 according to the invention may be comprised in an absorbent structure in an absorbent article 30, such as a diaper, a pant-type diaper, an incontinence garment, a sanitary napkin, a wipe, a towel, a tissue, a bed protector, a wound or sore dressing, a tampon-like product, or similar product. In normal use, an absorbent structure in such an absorbent article serves to absorb, retain and isolate body wastes or body exudates, e.g. urine, faeces, blood, menstruation blood, fluid matter from wounds and sores, rinsing fluid and saliva. When a sensing device 10 according to the invention, in which the magnetoelastic film 12 is at least partially coated with a layer comprising a wetness sensitive polymer or detector molecules, is comprised in such an absorbent structure it will enable easy detection of wetness or a biological and/or chemical analyte, i.e. it will enable easy detection of an event such as urination or defecation. The detection is performed by detecting a change of the resonant frequency of the sensing device 10, as described above. Thereby the status of the absorbent structure and, thus, of the absorbent article 30 may be easily monitored by a user, parent, caregiver, etc.

For example, a sensing device 10 according to the invention may replace the sensing device disclosed in WO 2004/021944 and thus be comprised in the absorbent structures and absorbent articles disclosed in WO 2004/021944. Thus, a sensing device 10 according to the invention may be positioned in different positions in an absorbent structure in accordance with the positions of the sensing device in WO 2004/021944 and an absorbent article may also comprise more than one sensing device 10. For example, an absorbent article may comprise 1-10 sensing devices 10 according to the invention.

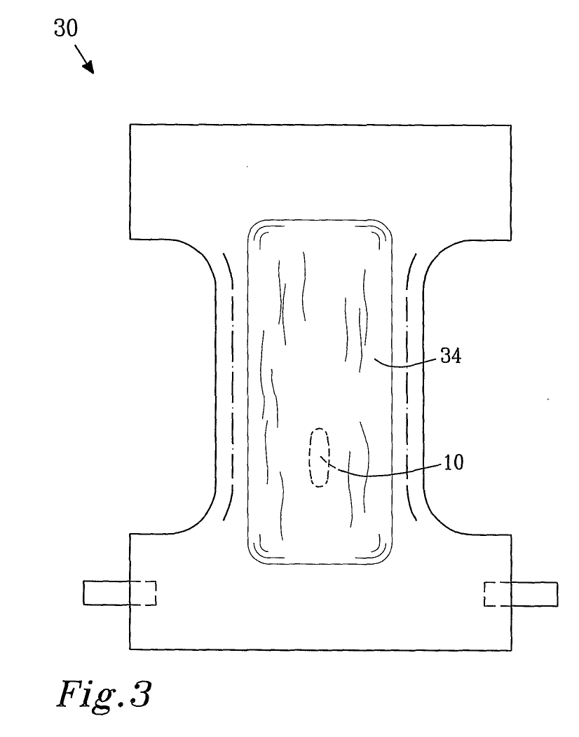

One non-limiting example of an absorbent article 30 comprising a sensing device 10 according to the invention is schematically shown in FIG. 3.

### EXAMPLES

### Bias Magnet

In a sensor based on magnetoelastic resonance, the magnetoelastic film (MER film) is mounted together with a bias magnet. The amplitude of the magnetic signal generated by the MER film and the MER film's resonance frequency is dependent on the strength of the bias field, see FIG. 1. It can be seen, that there is an optimal bias where the amplitude of the film is maximal. The resonance frequency reaches a minimum close to the optimal bias field. For some magnetoelastic alloys the amplitude maxima and the frequency minima coincide, but for the measured material 2826MB3, they are separated. It is desired to operate close to the frequency minimum, since the resonance frequency becomes less sensitive to small variations in the bias field. Variations can be due to for instance spread in bias magnet properties, variation in the position of the MER-film introduced at the mounting or different orientation of the sensor in the Earth's magnetic field.

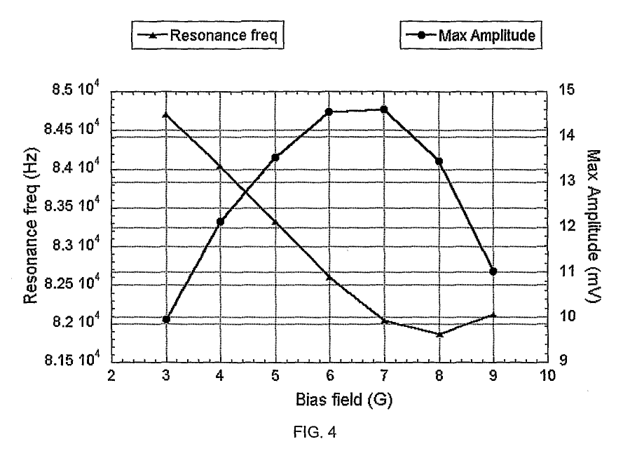

FIG. 4 shows the resonance frequency and amplitude versus the magnetic bias field. The measured MER-film was made from Metglas 2826MB3 with dimensions 30 mm×10 mm×25 um.

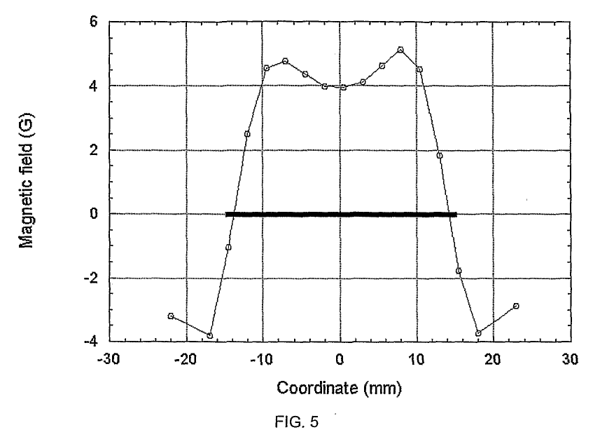

The measurement presented in FIG. 4 has been done in a uniform magnetic field generated by a Helmholtz coil. A uniform bias field is difficult to realize in a real sensor capsule. Usually the magnetoelastic film is placed close to, and in parallel with, a thin bias magnet with a shape and dimensions similar to the film. The field from such a bias magnet is shown in FIG. 5. The figure shows the magnetic field (only the component parallel to the length of the film) from the bias magnet at the location of MER film. The magnet has dimensions 35 mm×8 mm with the long axis edges cut at a 45 degree angle. For comparison with a typical MER-film, a 30 mm long line has been inserted. The edges of the bias magnet have been cut at an angle to reduce the filed non-uniformity at the edges, but the field is still far from uniform. It can bee seen that the field is rather uniform at the center on the film, but it decreases rapidly towards zero at the edges. This is typical for a bias magnet that has a length similar the MER film. The non-uniform field leads to that the film is not optimally biased at the edges and the effective bias will become more sensitive to the exact position of the MER-film in the capsule.

FIG. 5 shows the magnetic field (only the component parallel to the length of the film) from the bias magnet at the location of MER film. The magnet has dimensions 35 mm×8 mm with the long axis edges cut at a 45 degree angle. For comparison with a typical MER-film, a 30 mm long line has been inserted.

If the MER-film is mounted in the support 14 of the invention a more uniform bias field is obtained at the MER-film. This makes is easier to control the bias of the MER-film and reduces the spread in the bias due to misalignment at the mounting of the MER-film in the capsule. Another advantage of a longer bias magnet is that a longer bias magnet, and hence more uniform field, leads to less attraction between the MER-film and the bias magnet.

The MER film is magnetized by the magnetic field from the bias magnet. Due to the non-uniform bias field, the magnetized MER film will experience an attractive magnetic force in the regions close to the poles of the magnet. The MER film is bent or pressed against the material under the film. This may constrain the vibration of the film and/or cause extra friction against the material underneath the film. This may lower the amplitude of the signal generated by the MER film. A long bias magnet (in comparison to the film) moves the magnetic poles away from the film and the film will experience less attraction towards the bias magnet.

The present invention should not be limited by the embodiments and Figures described herein, but rather, the scope and limits of the invention are determined by the enclosed claims.

1. A sensing device, comprising a magnetoelastic film and a polymeric support for said magnetoelastic film, said magnetoelastic film being mounted in said support, the polymeric support comprises a mixture of polymeric material and magnetically-susceptible material, said magnetically-susceptible material being in particulate form.

2. The sensing device according to claim 1, wherein the polymeric support has a magnetic field, said magnetic field being oriented in a direction which is parallel to a plane of the magnetoelastic film.

3. The sensing device according to claim 1, wherein the magnetoelastic film is strip-shaped, with a longitudinal axis and a transverse axis, and has a greater extension in the longitudinal direction than the transverse direction, and the magnetic field of the polymeric support is oriented in a direction which is parallel to the longitudinal axis of the magnetoelastic film.

4. The sensing device according to claim 1, wherein the magnetoelastic film is fixedly mounted on the polymeric support only in the centre of the magnetoelastic film as measured in the longitudinal direction.

5. The sensing device according to claim 1, wherein the polymeric support comprises between 50 and 95 weight % of the magnetically-susceptible material.

6. The sensing device according to claim 1, wherein the polymeric material is selected from the group comprising polyamines, polyamides, polyethers, polyalkenes (e.g. polyethylene), polyacrylates, polyacrylamides, polystyrenes, polyvinylhalides, polyesters and mixtures thereof.

7. The sensing device according to claim 1, wherein the polymeric support is in the form of a capsule which completely encloses the magnetoelastic film, said capsule having a lid and a base and at least one opening which allows the passage of gases into the capsule.

8. The sensing device according to claim 7, wherein only the lid or the base of the capsule comprises the magnetically-susceptible material.

9. The sensing device according to claim 7, wherein the at least one opening in the capsule is covered with a liquid-impermeable, gas-permeable layer.

10. The sensing device according to claim 1, wherein the strength of magnetic field in the polymeric support lies between 0.1 and 2 mT.

11. A method for producing a sensing device according to claim 1, said method comprising:
a. mixing the polymeric material with the magnetically-susceptible material in particulate form;
b. casting the polymeric support from the mixture of the polymeric material and the magnetically-susceptible material;
c. magnetising the polymeric support so as to provide it with an overall magnetic field; and
d. mounting the magnetoelastic film on the polymeric support such that the plane of the magnetoelastic film is oriented in a direction which is parallel to the magnetic field.

12. The method according to claim 11, further including
e. calibrating the sensing device after the magnetoelastic film has been mounted on the polymeric support.

13. The method according to claim 11, wherein the polymeric support comprises a capsule, having a lid and a base and at least one opening which allows the passage of gases, wherein step d. comprises the individual steps of mounting the magnetoelastic film in the base of the capsule such that the plane of the magnetoelastic film is oriented in a direction which is parallel to the magnetic field of the capsule and closing the lid so that the magnetoelastic film is completely enclosed within the capsule.

14. (canceled)

15. An absorbent article, comprising one or more sensing devices according to claim 1.

16. A method of detecting wetness comprising providing a sensing device according to claim 1 in an absorbent article and monitoring the sensing device.

17. The sensing device according to claim 1, wherein the polymeric support comprises between 75 and 90 weight % of the magnetically-susceptible material.

18. The sensing device according to claim 1, wherein the strength of magnetic field in the polymeric support lies between 0.1 and 0.5 mT.

19. The absorbent article of claim 15, wherein the absorbent article is a diaper, incontinence guard, sanitary napkin or panty liner.

20. The method of claim 16, wherein the absorbent article is a diaper, incontinence guard, sanitary napkin or panty liner.
